(19) United States
(12) Patent Application Publication
(10) Pub. No.: US 2025/0057778 A1
(43) Pub. Date: Feb. 20, 2025

(54) BIOABSORBABLE PARTICLES AND METHOD OF USE

(71) Applicants: W. L. Gore & Associates, Inc., Newark, DE (US); JANSSEN PHARMACEUTICA NV, Beerse (BE); Janssen Research & Development, LLC, Raritan, NJ (US)

(72) Inventors: Robert L. Cleek, Flagstaff, AZ (US); Mei Li, Middletown, DE (US); Kayla Stewart, Flagstaff, AZ (US); Donna KLEIN, Spring House, PA (US); Hoang Dung LU, Spring House, PA (US)

(21) Appl. No.: 18/721,374

(22) PCT Filed: Dec. 20, 2022
(86) PCT No.: PCT/US2022/053491

§ 371 Date: Jun. 18, 2024

Related U.S. Application Data
(60) Provisional application No. 63/268,790, filed on Mar. 2, 2022.
(60) Provisional application No. 63/265,732, filed on Dec. 20, 2021.

(51) Int. Cl. A61K 9/51; A61K 31/702

(52) U.S. Cl. CPC A61K 9/5153; A61K 9/5192; A61K 31/702

(57) ABSTRACT

The present disclosure provides bioabsorbable particles with a nucleic acid. The bioabsorbable particles also include a bioabsorbable polymer matrix and a lipid salt. both of which can be altered to affect the particle properties. The bioabsorbable particles may be administered to a patient for the treatment of diseases. Methods for making and using the bioabsorbable particles are also provided.

### CROSS-REFERENCE TO RELATED APPLICATION

This application is a national phase application of PCT Application No. PCT/US2022/053491, internationally filed on Dec. 20, 2022, which claims the benefit of Provisional Application No. 63/268,790, filed Mar. 2, 2022, and also claims the benefit of Provisional Application No. 63/265,732, filed Dec. 20, 2021, which are herein incorporated by reference in their entireties for all purposes.

### FIELD

The present disclosure relates generally to bioabsorbable particles. More specifically, the disclosure relates to particles that include a polymer, a lipid salt, and a nucleic acid agent that are used for medical treatment.

### BACKGROUND

Numerous developments in the medical field have led to new technologies for combatting diseases. One such development is the use of active agents involving nucleic acids to treat inflammatory diseases, such as Inflammatory Bowel Disease (IBD). However, delivering active agents to the colon for treatment of IBD can be difficult due to physiological challenges, biochemical barriers, and environmental barriers, including those associated with mucus and epithelium.

### SUMMARY

In one aspect (“Aspect 1”), a formulation includes a plurality of bioabsorbable particles, each bioabsorbable particle including a polymer matrix, an antisense oligonucleotide (ASO) encapsulated within the polymer matrix, and a cationic lipid encapsulated within the polymer matrix where the plurality of bioabsorbable particles contains at least about 1 weight percent of antisense oligonucleotide (ASO).

In another aspect (“Aspect 2”), further to Aspect 1, the plurality of bioabsorbable particles have an average particle size of less than 300 nm.

In another aspect (“Aspect 3”), further to any one of Aspects 1-2, the plurality of bioabsorbable particles contain at least about 5 weight percent of the ASO.

In another aspect (“Aspect 4”), further to any one of Aspects 1-3, the plurality of bioabsorbable particles contain at least about 10 weight percent of the ASO.

In another aspect (“Aspect 5”), further to any one of Aspects 1-4, the plurality of bioabsorbable particles contain at least about 15 weight percent of the ASO.

In another aspect (“Aspect 6”), further to any one of Aspects 1-5, the polymer matrix includes trimethylene carbonate (TMC).

In another aspect (“Aspect 7”), further to any one of Aspects 1-6, the polymer matrix comprises a copolymer of TMC and polylactic acid (PLA).

In another aspect (“Aspect 8”), further to Aspect 7, the ratio of TMC to PLA is about 25:75.

In another aspect (“Aspect 9”), further to any one of Aspects 1-8, the polymer matrix comprises a poly(lactic-co-glycolic acid) (PLGA) copolymer.

In another aspect (“Aspect 10”), further to any one of Aspects 1-9, including a surface polymer coupled to a surface of each of the bioabsorbable particles.

In another aspect (“Aspect 11”), further to Aspect 10, the surface polymer comprises polyethylene glycol (PEG).

In another aspect (“Aspect 12”), further to Aspect 11, the bioabsorbable particles contain at least 5 weight percent of the PEG.

In another aspect (“Aspect 13”), further to any one of Aspects 1-12, the plurality of bioabsorbable particles contain about 10 weight percent to about 40 weight percent of the cationic lipid.

In another aspect (“Aspect 14”), further to any one of Aspects 1-13, the plurality of bioabsorbable particles contain about 20 weight percent to about 30 weight percent of the cationic lipid.

In another aspect (“Aspect 15”), further to any one of Aspects 1-14, the plurality of bioabsorbable particles comprise nanoparticles.

In another aspect (“Aspect 16”), further to any one of Aspects 1-14, the plurality of bioabsorbable particles comprise microparticles.

In one aspect (“Aspect 17”), a method of treating a patient in need thereof includes delivering to the patient a formulation according to any of Aspects 1-16 and allowing sufficient time so that the polymer matrix to degrade in the patient, and release the nucleic acid within the patient.

In one aspect (“Aspect 18”), a method of making a formulation of bioabsorbable particles includes dissolving a structural polymer and a cationic lipid in a solvent, adding a nucleic acid of 2 to 75 nucleotides in length to the dissolved structural polymer and cationic lipid, sonicating the mixture of structural polymer, cationic lipid, and nucleic acid in a first sonication to create a water-in-oil emulsion, adding an aqueous solution to the water-in-oil emulsion, sonicating the water-in-oil emulsion in a second sonication to produce a water-in-oil-in-water emulsion, adding water to the water-in-oil-in-water emulsion, collecting a plurality of particles from the water-in-oil-in-water emulsion, and cleaning the plurality of particles.

In another aspect (“Aspect 19”), further to Aspect 18, the cationic lipid and structural polymer are dissolved in a ratio of about 1:4 by mass to about 1:1 by mass.

In another aspect (“Aspect 20”), further to Aspect 18 or 19, the nucleic acid is an antisense oligonucleotide (ASO) and is loaded into the plurality of particles at about 5 weight percent to about 20 weight percent.

In another aspect (“Aspect 21”), further to any one of Aspects 18-20, the first sonication and the second sonication are completed in an ice bath.

In another aspect (“Aspect 22”), further to any one of Aspects 18-21, the aqueous solution is a polyvinyl alcohol solution.

In another aspect (“Aspect 23”), further to any one of Aspects 18-22, the step of collecting the plurality of particles from the emulsion is completed through centrifugation.

In another aspect (“Aspect 24”), further to any one of Aspects 18-23, including lyophilizing the oil solution in the presence of a sugar and a buffer.

In another aspect (“Aspect 25”), further to Aspect 24, the sugar is trehalose.

In another aspect (“Aspect 26”), further to Aspect 24 or 25, the buffer is histidine.

In one aspect (“Aspect 27”), a method of treating a patient in need thereof includes (1) delivering a plurality of particles to the patient where each particle includes a polymer matrix, a nucleic acid of 2 to 75 nucleotides in length encapsulated within the polymer matrix, and a cationic lipid encapsulated within the polymer matrix wherein the ratio of cationic lipid to the polymer matrix in each particle is from about 1:4 by mass to about 1:1 by mass and (2) allowing sufficient time to pass so that the polymer matrix biodegrades in the patient and release the nucleic acid.

In another aspect (“Aspect 28”), further to Aspect 27, the nucleic acid is an antisense oligonucleotide (ASO) loaded in the plurality of particles at from about 5 weight percent to about 20 weight percent, and the cationic lipid is one of 1,2-di-O-octadecenyl-3-trimethylammonium propane chloride salt (DOTMA) or 1,2-dioleoyl-3-trimethylammonium-propane chloride salt (DOTAP).

In another aspect (“Aspect 29”), further to Aspect 27 or 28, the polymer matrix biodegrades in a colon of the patient.

In another aspect (“Aspect 30”), further to any one of Aspects 27-29, each particle further comprises a surface polymer on the surface of each particle, the surface polymer comprising polyethylene glycol (PEG).

In another aspect (“Aspect 31”), further to Aspect 30, each particle comprises at least 5 wt. % of the PEG.

In another aspect (“Aspect 32”), further to any one of Aspects 27-31, the plurality of particles comprises nanoparticles.

In another aspect (“Aspect 33”), further to any one of Aspects 27-32, the patient has at least one of inflammatory bowel disease, cardiovascular disease, hypertension, chronic heart failure, diabetes, Parkinson's disease, prostate cancer, and chronic kidney disease.

In one aspect (“Aspect 34”), a formulation includes a plurality of bioabsorbable particles, each bioabsorbable particle includes a polymer matrix, a nucleic acid encapsulated within the polymer matrix, a cationic lipid encapsulated within the polymer matrix, and a surface polymer coupled to a surface of each of the bioabsorbable particles, where the surface polymer includes polyethylene glycol (PEG) and each of the bioabsorbable particles includes at least 5 weight percent of the PEG.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic representation of a bioabsorbable nanoparticle in accordance with at least one embodiment.

FIG. 2 is a schematic representation of a bioabsorbable nanoparticle that includes a surface polymer in accordance with at least one embodiment.

FIG. 3 is a flowchart for a method of making the bioabsorbable nanoparticle of FIG. 1 in accordance with at least one embodiment.

FIG. 4 is a partial flowchart similar to FIG. 3 for making the bioabsorbable nanoparticle of FIG. 2 in accordance with at least one embodiment.

FIG. 5 is a flowchart for a method of treating a patient with the bioabsorbable nanoparticle of FIGS. 1-2 in accordance with at least one embodiment.

FIG. 6 is a graphical illustration showing the relationship between lipid loading level and nanoparticle diameter in accordance with at least one embodiment.

FIG. 7 is a graphical illustration showing the relationship between lipid level and the nucleotide loading in accordance with at least one embodiment.

FIG. 8 is a graphical illustration showing the relationship between the lipid level and the zeta potential in accordance with at least one embodiment.

FIGS. 9 and 10 are graphical illustrations showing the relationship between incubation and nucleotide release for multiple trials in accordance with at least one embodiment.

FIG. 11 shows particle distributions of TFF cleaned nanoparticles, reconstructed particles in deionized water (di-water) and phosphate buffered saline (PBS) in accordance with at least one embodiment.

FIG. 12 is a gel assay as discussed in the Examples in accordance with at least one embodiment.

FIG. 13 is a scanning electron micrograph (SEM) image of bioabsorbable nanoparticles formed in accordance with at least one embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

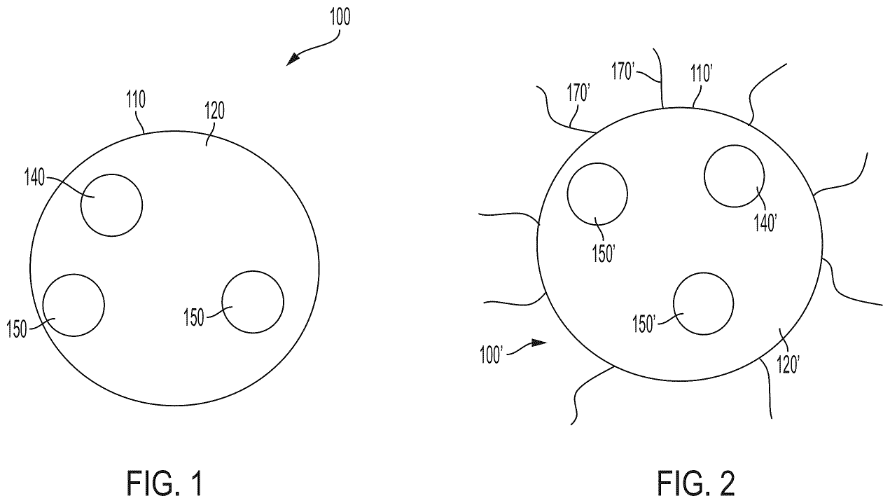

Referring first to FIG. 1, an illustration of exemplary bioabsorbable particle, nanoparticle 100, is shown. The nanoparticle 100 comprises a surface 110, a polymer matrix 120, a lipid 140, and a nucleic acid 150. While lipid 140 and nucleic acid 150 are illustrated as separate from one another within the nanoparticle 100, it is understood that lipid 140 is preferably complexed with nucleic acid 150 within nanoparticle 100. In an exemplary embodiment, nanoparticle 100 has a size of about 300 nm or less. In another exemplary embodiment, nanoparticle 100 has a size of about 100 nm to about 300 nm. In a most preferred embodiment, nanoparticle 100 has a size of about 200 nm to about 300 nm. Although a nanoparticle 100 is shown and described herein, the present disclosure also relates to microparticles having a size greater than about 300 nm, such as about 500 nm, 750 nm, 1000 nm (1 μm), or larger. Also, the nanoparticle 100 may have a neutral or near-neutral electrical charge, such as a zeta potential from about −20 mv to about +20 mv. Alternatively, the nanoparticle 100 may a zeta potential from about −10 mv to about +10 mv. Preferably the neutral or near-neutral nanonparticle 100 may have a zeta potential, of about 0 mv.

It should be noted that FIG. 1 is only a representation of a nanoparticle 100, and the depiction of a lipid 140 and nucleic acid 150 as heterogeneous features within the polymer matrix 120 is for illustrative purposes only. The polymer matrix 120, the lipid 140, and the nucleic acid 150 may be entirely or partially homogeneous. Alternatively, the polymer matrix 120, the lipid 140, and the nucleic acid 150 may be entirely or partially heterogenous. The polymer matrix 120, the lipid 140, and the nucleic acid 150 may be entirely or partially an emulsion. The polymer matrix 120, the lipid 140, and the nucleic acid 150 may be entirely or partially a suspension. The polymer matrix 120, the lipid 140, and the nucleic acid 150 may be entirely or partially any other form of mixture as understood and appreciated by those of skill in the art. Additionally, the polymer matrix 120, the lipid 140, and the nucleic acid 150 may be any state of matter. For example, the nucleic acid 150 may be a solid or a liquid encapsulated within the polymer matrix 120 (e.g. solid polymer matrix). Each of the polymer matrix 120, the lipid 140, and the nucleic acid 150 is described further below.

In a non-limiting embodiment, the polymer matrix 120 of the nanoparticle 100 is a bioabsorbable or biodegradable polymer. The polymer matrix 120 may be hydrophobic in nature to promote slower degradation than a hydrophilic matrix. The polymer matrix 120 may also exhibit low acid degradation to minimize inflammation. In one embodiment, the polymer matrix 120 may be a trimethylene carbonate (TMC)-based polymer, such as a copolymer of TMC and polylactic acid (PLA), hereinafter “PLA:TMC”. The PLA:TMC copolymer may be synthesized using methods well-known to the art, such as, for example, by combining TMC monomers with suitable comonomers of lactic acid, such as L-Lactic acid comonomers creating poly(L,Lactic acid-TMC) hereinafter “L-PLA:TMC”; D-Lactic acid comonomers creating poly(D,Lactic acid-TMC) hereinafter “D-PLA:TMC”; and comonomers of L-lactic acid and D-lactic acid and TMC creating Poly(DL,Lactic acid-TMC) hereinafter “D,L-PLA:TMC”. The PLA:TMC copolymers may have a weight ratio of D-PLA to TMC from 55% to 45% (55:45) or from 75% to 25% (75:25), a weight ratio of L-PLA to TMC from 55% to 45% (55:45) or from 75% to 25% (75:25), and a weight ratio of D,L-PLA to TMC from 50% to 50% (50:50) or from 75% to 25% (75:25) (all based on weight). In some aspects, the PLA:TMC copolymer may comprise from 45 to 60 wt. % PLA and from 40 to 55 wt. % TMC. In another embodiment, the polymer matrix 120 may comprise a terpolymer of PLA, TMC, and another polymer, such as polyethylene glycol (PEG), hereinafter “PLA:TMC:PEG”. In yet another embodiment, the polymer matrix 120 may comprise a poly(lactic-co-glycolic acid) (PLGA) copolymer containing PLA and polyglycolic acid (PGA) comonomers.

The type of polymer(s) used, the ratios of polymers used, and the method of making the polymer matrix 120 may all be altered to adjust the properties of the polymer matrix 120. Such properties may include: the ability of the polymer matrix 120 to degrade or otherwise release the nucleic acid 150, the effect of pH on the polymer matrix 120, the size of the nanoparticle 100, the amount of the lipid 140 and the nucleic acid 150 loaded into nanoparticle 100, the physical properties of the nanoparticle 100 (e.g., density, melting point, glass transition temperature, modulus, etc.), and the chemical properties of the nanoparticle 100 (e.g., molecular weight, polarity, charge, etc.). In an embodiment, the polymer matrix 120 is formulated to degrade within the gastrointestinal tract of a patient. In particular, the polymer matrix 120 may be formulated to degrade within the colon of the patient.

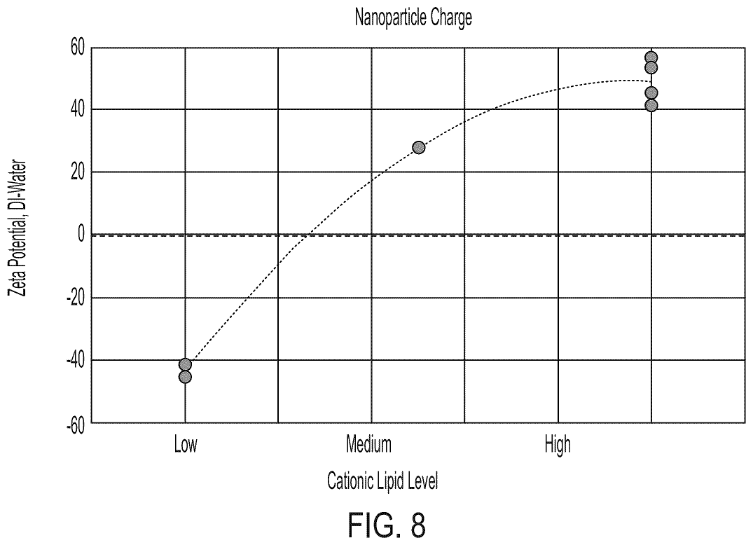

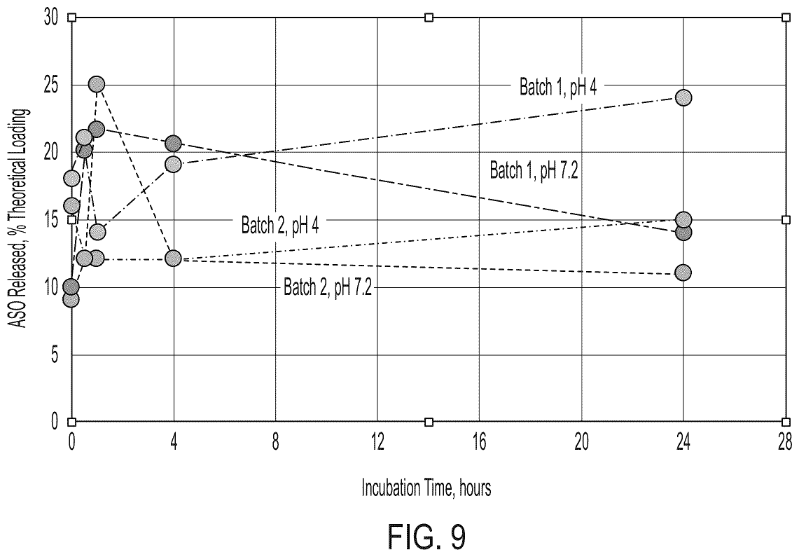

In one embodiment, the lipid 140 of the nanoparticle 100 is a cationic lipid salt. In particular, lipid 140 may be 1,2-dioleoyl-3-trimethylammonium-propane chloride salt (DOTAP) or 1,2-di-O-octadecenyl-3-trimethylammonium propane chloride salt (DOTMA). In other embodiments, any lipid 140 may be used. The nanoparticle 100 may be loaded with from approximately 10 wt % to approximately 40 wt % of the lipid 140 or from approximately 20 wt % to approximately 30 wt % of the lipid 140, although the amount of lipid 140 loaded in the nanoparticle 100 may be altered to adjust characteristics of nanoparticle 100, such as size, loading and/or release of nucleic acid 150, charge, zeta potential, etc. The loading of the lipid 140 may also be expressed as a ratio with respect to the amount of the polymer matrix 120. The nanoparticle 100 may be loaded with the lipid 140 such that the ratio of the lipid 140 to the polymer matrix 120 is approximately 1:4 by mass. The nanoparticle 100 may be loaded with the lipid 140 such that the ratio of the lipid 140 to the polymer matrix 120 is approximately 1:1 by mass. The nanoparticle 100 may be loaded with the lipid 140 such that the ratio of the lipid 140 to the polymer matrix 120 is approximately 1:2 by mass. The nanoparticle 100 may be loaded with the lipid 140 such that the ratio of the lipid 140 to the polymer matrix 120 is approximately 3:4 by mass. The loading of the lipid 140 may also be expressed as a ratio with respect to the amount of the nucleic acid 150. The nanoparticle 100 may be loaded with the lipid 140 such that the ratio of the lipid 140 to the nucleic acid 150 is approximately 1:1 by mass. The nanoparticle 100 may be loaded with the lipid 140 such that the ratio of the lipid 140 to the nucleic acid 150 is approximately 2:1 by mass. The nanoparticle 100 may be loaded with the lipid 140 such that the ratio of the lipid 140 to the nucleic acid 150 is approximately f 5:4 by mass. The nanoparticle 100 may be loaded with the lipid 140 such that the ratio of the lipid 140 to the nucleic acid 150 is approximately 7:4 by mass. The nanoparticle 100 may be loaded with the lipid 140 such that the ratio of the lipid 140 to the nucleic acid 150 is approximately 3:2 by mass. The lipid 140 loading allows for more fine-tuning of nanoparticle 100 characteristics. Generally, an increase in the amount of the lipid 140 loaded in the nanoparticle 100 corresponds to an increase in loading of the nucleic acid 150, and an increase in zeta potential (FIGS. 7-8). It is typically desirable for the nanoparticle 100 to have a neutral or slightly negative charge, which can be altered by the loading and type of the lipid 140 in the nanoparticle 100.

The nucleic acid 150 of the nanoparticle 100 comprise short-chain nucleotides. In some embodiments, the nucleic acid 150 is an oligonucleotide. Oligonucleotides are short DNA and/or RNA molecules, and/or oligomers, that have a wide range of applications in disease treatment, genetic testing, and research. The use of oligonucleotides as therapeutic agents rests upon their ability to interfere, in a sequence-specific manner, with the fundamental machinery of protein synthesis either by binding to the mRNAs transcribed from a gene or by binding directly to a target gene. The oligonucleotide may be an antisense oligonucleotide (ASO). ASO's can be used to regulate gene expression and to treat inflammatory and neoplastic diseases.

Oligonucleotides are characterized by the sequence and number of nucleotide residues that make up the entire molecule. The number of DNA and/or RNA nucleotide units that make up the molecule usually denotes the length of the oligonucleotide. For example, an oligonucleotide of 10 nucleotides, or bases, is a “10-mer” oligonucleotide. Often chemical modification of the backbone or sugar ring is performed to, for example, increase nuclease resistance or specificity to the target.

Nucleic acids of less than 75 nucleotides are preferred in order to avoid shearing of the nucleic acid during the sonication steps used to create the bioabsorbable particles. More preferred, the nucleic acids are of less than 40 nucleotides.

The nanoparticle 100 may be loaded with at least 5 wt % of the nucleic acid 150, approximately 5 wt %, approximately 10 wt %, approximately 15 wt %, approximately 20 wt %, approximately 25 wt %, approximately 30 wt %, or more of the nucleic acid 150, although this amount may be altered to adjust characteristics of the nanoparticle 100.

The nanoparticle 100 is configured to degrade within the GI tract of a patient and to release the nucleic acid 150 for medical treatment. In one example, the nanoparticle 100 is configured to be absorbed/degraded within the colon of the patient, and the nucleic acid 150 can therefore be delivered directly within the colon to treat an inflammatory bowel disease (e.g. Crohn's disease, ulcerative colitis, etc.). Further, the nanoparticles 100 may be configured to degrade, be absorbed, or release an active agent within any organ of the patient or at any point along the GI tract (e.g. stomach, intestines). While in the illustrated embodiments the nanoparticles 100 are loaded with a nucleic acid 150 suitable for treatment of inflammatory bowel disease, in other embodiments the nanoparticles 100 may be loaded with nucleic acids 150 or any active ingredient or drug suitable for treatment of a wide variety of medical issues including, but not limited to, cardiovascular disease, hypertension, chronic heart failure, diabetes, Parkinson's disease, prostate cancer, and chronic kidney disease.

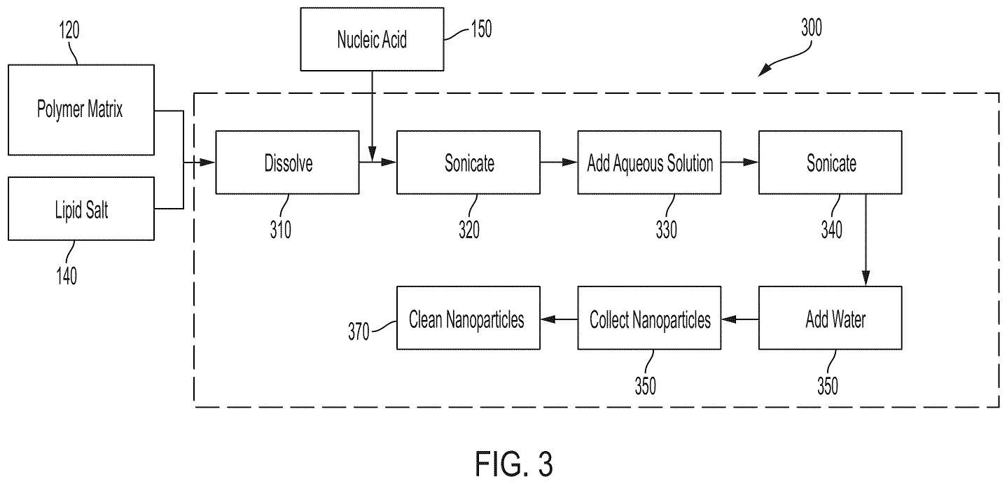

Referring next to FIG. 2, another exemplary nanoparticle 100′ is shown. Nanoparticle 100′ of FIG. 2 is similar to nanoparticle 100 of FIG. 1, with like reference numerals identifying like elements, except as described herein. As shown in FIG. 2, the nanoparticle 100′ may also comprise a surface polymer 170′. In one embodiment, the surface polymer 170′ is covalently bonded to at least the surface 110′ of the nanoparticle 100′. A non-limiting example of a surface polymer 170′ is a copolymer of PLA and polyethylene glycol (PEG). When PEG is used as the surface polymer 170′ (either on its own or in a copolymer), the surface 110′ may be described as being “PEGylated”. The surface polymer 170′ may be added onto the surface 110′ of the nanoparticle 100′ by forming the nanoparticle 100′ in the presence of a physical blend of the surface polymer 170′, or by forming terpolymers with the surface polymer 170′ and the polymer matrix 120′. Additionally, the surface polymer 170′ may be added to the surface 110′ through a coupling group located on the nanoparticle 100′. Non-limiting examples of such coupling groups include 1-ethyl-3-(3-dimethylaminopropyl)carbodiimide (EDC), succinimidyl active esters, and NH₂-PEG. In other embodiments, the surface polymer 170′ may be added onto the surface 110′ after the nanoparticle 100′ has been made. The surface polymer 170′ may assist in the migration of the nanoparticles 100′, in the bioabsorption of the nanoparticles 100′, in the degradation of the nanoparticles 100′, or in altering the physical or chemical characteristics of the nanoparticles 100′. Each nanoparticle 100′ may include at least about 1 wt. %, at least about 5 wt. %, at least about 10 wt. %, at least about 15 wt. %, or at least about 20 wt. % of the surface polymer 170′. In some embodiments, each nanoparticle may include from about 1 wt. % to about 20 wt. %, from about 1 wt. % to about 15 wt. %, from about 1 wt. % to about 10 wt. %, from about 5 wt. % to about 20 wt. %, from about 5 wt. % to about 15 wt. %, or from about 5 wt. % to about 10 wt. %.

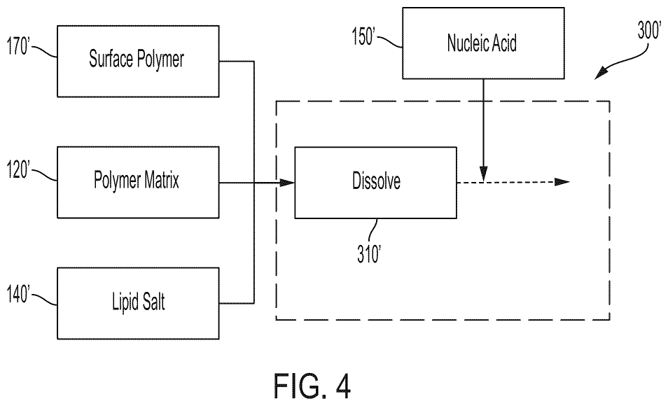

Referring now to FIG. 3, a method of making 300 the nanoparticle 100 of FIG. 1 is shown. The method of making 300 includes dissolving 310 in the structural polymer matrix 120 and the lipid salt 140 in a suitable solvent, adding the nucleic acid 150 and sonicating 320 the mixture to produce a water-in-oil emulsion, adding an aqueous solution 330 such as polyvinyl alcohol, sonicating 340 the resulting mixture to produce a water-in-oil-in water-in-oil emulsion, adding water 350, collecting 360 the nanoparticles 100 by drying, centrifuging, or another suitable method, and cleaning 370 the nanoparticles 100. While this method demonstrates one way to make the nanoparticles 100, additional steps may be added to the method 300 including, such as, slowly shaking water-in-oil emulsion for a few hours, stirring the double emulsion overnight after adding water 350, dispersing the nanoparticles 100 in water after cleaning 370, and/or lyophilizing the nanoparticles 100 after cleaning 370. The step of dissolving 310 may be completed with any solvent capable of effectively dissolving the polymer matrix 120 and the lipid 140, such as, but not limited to, methylene chloride. The steps of sonicating 320, 340 may be carried out over an ice bath. In other embodiments, the sonication may take place at various temperatures, over various lengths of time, and with various amplitudes. See Example 7 for an embodiment of the method of making 300. Once the nanoparticles 100 have been made, they can be analyzed as is known in the art and as discussed in the later examples.

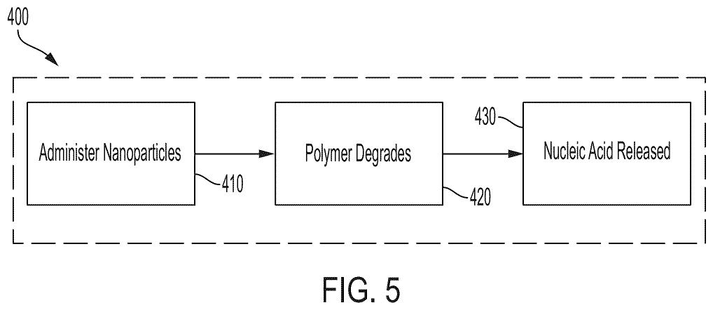

Referring now to FIG. 4, a method of making 300′ the nanoparticle 100′ of FIG. 2 is shown in part. The method of making 300′ shown in FIG. 4 is similar to the method of making 300 of FIG. 3, with like reference numerals identifying like elements, except as described herein. As shown in FIG. 4, the surface polymer 170′ is added to the polymer matrix 120′ and the lipid salt 140′ during the dissolving 310′.

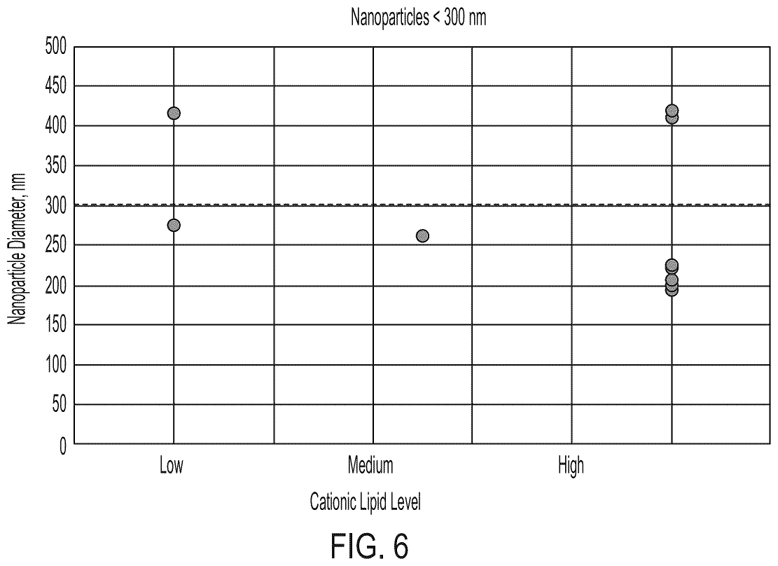

Referring now to FIG. 5, a method for treatment 400 is shown. This method of treatment 400 is described with respect to the nanoparticles 100 of FIG. 1, but this method of treatment 400 is equally applicable to the nanoparticles 100′ of FIG. 2. The method of treatment 400 includes administering 410 the nanoparticles 100 to the patient, wherein the polymer matrix 120 will degrade 420 in the patient, and subsequently releasing 430 the nucleic acid 430 from the nanoparticle 100 and into the patient. The nanoparticles 100 may be administered 410 orally through a pill, tablet, or solution, or it may be administered 410 through injection. If administered orally, the nanoparticles 100 may be located initially in a capsule (not shown) that degrades at a point or specific region in the GI tract. In one embodiment, the capsule has an enteric coating that allows for the degradation of the capsule upon a change in pH.

The nanoparticles 100 are configured to be bioabsorbable such that the polymer matrix 120, the lipid 140, and/or the nucleic acid 150 may be absorbed by the body. One potential method of absorption is through pinocytosis within cells. In pinocytosis, the cell membrane extends and folds around extracellular material, forming a pouch that creates an internalized vesicle. The vesicles eventually fuse with the lysosome where the contents (in this case, the nanoparticles 100) are digested. In this way, the polymer matrix 120 may degrade within cells in the GI tract through the cell's internal digestion process, and the nucleic acid 150 will be delivered to the cells thereafter. In this way, the nucleic acid 150 can be delivered to targeted areas of the patient to treat various ailments, such as irritable bowel syndrome, cardiovascular disease, hypertension, chronic heart failure, diabetes, Parkinson's disease, prostate cancer, or chronic kidney disease.

The nanoparticles 100, 100′ shown in FIGS. 1-2 and the methods shown in FIGS. 3-5 are provided as examples of the various features of the device/methods and, although the combination of those illustrated features is clearly within the scope of invention, that example and its illustration is not meant to suggest the inventive concepts provided herein are limited from fewer features, additional features, or alternative features to one or more of those features shown in FIGS. 1-5. For example, in various embodiments, the steps of the method shown in FIG. 5 may include the steps described with reference to FIG. 3. It should also be understood that the reverse is also true. In addition, one or more of the components depicted in FIG. 1 can be employed in addition to, or as an alternative to components depicted in FIG. 2.

### TEST METHODS

It should be understood that although certain methods and equipment are described below, other methods or equipment determined suitable by one of ordinary skill in the art may be alternatively utilized.

### Inherent Viscosity

Inherent viscosity (IV) was measured using a Cannon MiniPV-HX Automatic Viscometer with hexafluoroisopropanol (HFIP) as the solvent for extraction.

### Nanoparticle Size

Nanoparticle size was measured using an Ultrasizer (Marlven Panalytical) at multiangle light scattering mode.

### Electrical Charge

Zeta potential of the nanoparticle dispersion was measured using an Ultrasizer (Marlven Panalytical) at multiangle light scattering mode.

### Oligonucleotide Release

The amount and rate of oligonucleotide released from the nanoparticles was measured through gel electrophoresis as detailed in Example 9.

### PEG Density Characterization

The amount of PEG content on nanoparticle surface was measured through a UV assay as detailed in Example 21.

### EXAMPLES

### Polymer Synthesis

The bioabsorbable polymers identified in Examples 1-5 were synthesized according to the methodologies described in “Analysis and characterization of resorbable DL-lactide trimethylene carbonate copolyesters”, Journal of Material Science: Materials in Medicine 4 (1993) pp. 381-88.

### Example 1: L-PLA:TMC Copolymer Characterization

This example describes the copolymer of L-PLA:TMC in a ratio of 75:25. Table 1 shows the characterization data for the composition in this example, in the form of a purified sample. To obtain the purified sample, the copolymer of L-PLA:TMC was dissolved in chloroform (CHCl₃) at 2-5 wt %, was precipitated in 10× isopropyl alcohol (IPA), and then dried in vaccuo. For this example, IV (HFIP)=1.349 dL/g and the glass transition for the polymer was determined to be 32.95° C.

### Example 2: D,L-PLA:TMC-PEG5K Copolymer Characterization

This example describes the copolymer D,L-PLA:TMC-PEG5K in a ratio of 86.4:8.7:4.9 Table 1 shows the characterization data for the composition. For this example, IV (HFIP)=1.3 dL/g and the glass transition for the polymer was determined to be 31.7° C.

### Example 3: D,L-PLA:TMC-PEG5K Copolymer Characterization

This example describes the copolymer D,L-PLA:TMC-PEG5K in a ratio of 73.4:21.8:4.8 Table 1 shows the characterization data for the composition. For this example, IV (HFIP)=1.2 dL/g and the glass transition for the polymer was determined to be 20.4° C.

### Example 4: D,L-PLA:TMC-PEG5K Copolymer Characterization

This example describes the copolymer D,L-PLA:TMC-PEG5K in a ratio of 82:8:10. Table 1 shows the characterization data for the composition. For this example, IV (HFIP)=1.0 dL/g and the glass transition for the polymer was determined to be 26.2° C.

### Example 5: D,L-PLA:TMC-PEG10K Copolymer Characterization

This example describes the copolymer D,L-PLA:TMC-PEG10K in a ratio of 86.5:8.5:5 Table 1 shows the characterization data for the composition. For this example, IV (HFIP)=1.4 dL/g and the glass transition for the polymer was determined to be 34.5° C.

TABLE 1

| | L-PLA-TMC | D,L-PLA-TMC- | D,L-PLA-TMC- | D,L-PLA-TMC- | D,L-PLA-TMC- |
|---|---|---|---|---|---|
| Sample ID | purified | PEG5K | PEG5K | PEG5K | PEG10K |
| Monomer | 75:25 | 86.4:8.7:4.9 | 73.4:21.8:4.8 | 82:8:10 | 86.5:8.5:5 |
| weight ratio | | | | | |
| 67IV (dL/g) | 1.349 | 1.2 | 1.4 | 1.0 | 1.3 |
| Tg (° C.) | 32.95 | 20.4 | 34.5 | 26.2 | 31.7 |
| Mn (g/mol) | 40,764 | 9,424 | 11,690 | 9,705 | 6,771 |
| Mw(g/mol) | 91,592 | 31,293 | 36,359 | 22,271 | 36,652 |
| Mz (g/mol) | 132,722 | 78,229 | 91,486 | 44,451 | 109,087 |
| PD | 2.25 | 3.32 | 3.11 | 2.29 | 5.41 |
| Example | 1 | 2 | 3 | 4 | 5 |

### Example 6: Oligonucleotide Descriptions

This example describes oligonucleotides utilized in the examples for nanoparticle encapsulation.

The oligonucleotide used in nanoparticle encapsulation that is termed “Oligo-1” is a 20-mer oligonucleotide with 5 nucleotides 2′ O-methylation on each end of the oligonucleotide.

The oligonucleotide used in nanoparticle encapsulation that is termed “Oligo-2” is C*G*A* C*G*C* C*C*C* T*C*T* T*C*C* C*C*G* C*T*G with no methylation and the stars indicating phosphorothioated bonds.

The oligonucleotide used in nanoparticle encapsulation that is termed “Oligo-3” is mC*mG*mA* mC*mG*C* C*C*C* T*C*T* T*C*C* C*mC*mG* mC*T*mG with methylation indicated by “m” and stars indicating phosphorothioated bonds.

### Example 7: Nanoparticles of Oligo-1 in L-PLA:TMC Copolymer with Low Level of Lipid Salt DOTAP

This example demonstrates encapsulation of Oligo-1 in L-PLA:TMC copolymer nanoparticles in the presence of low level of lipid salt DOTAP, specifically 19 wt. % of lipid salt DOTAP and a 1:1 ratio of lipid salt DOTAP to Oligo-1, as described herein.

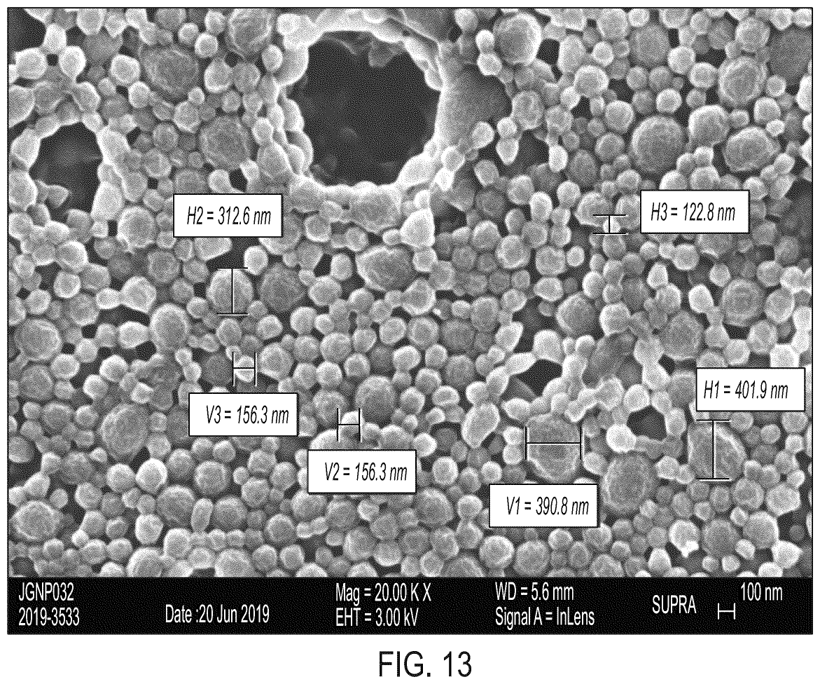

Nanoparticles were prepared by dissolving approximately 160 mg of L-PLA:TMC 75:25 (“LT-75”) polymer (as described in Example 1) and 50 mg of lipid salt DOTAP (1,2-dioleoyl-3-trimethylammonium-propane chloride salt, Avanti Polar Lipids Inc) in 6 mL methylene chloride at room temperature. 0.5mL of the 100 mg/ml of Oligo-1 aqueous solution (in DNase/RNase-Free distilled water, Invitrogen) was pipetted into the polymer/DOTAP oil solution. The mixture was sonicated for 1 min over ice water bath using a Branson SXF150 sonifier equipped with a 1/4″ tapered probe. Continuous mode was used with 45% of amplitude for sonication. The water-in-oil (W/O) emulsion was placed on a shaker at a speed of 100 rpm/min overnight. The separated mixture was sonicated again for 1 min over ice water to generate a good dispersion. Approximately 15 mL of 5% of polyvinyl alcohol (Aldrich, Mowiol 4-88) aqueous solution was then added to the dispersion. The resultant mixture was sonicated for 2 min using the same sonifier and probe over an ice water bath. Continuous mode was used with 45% of amplitude for sonication to produce an oil in water emulsion. Next, 230 mL of deionized water was added to the emulsion under magnetic stirring at 500 rpm. The emulsion was stirred overnight at room temperature to remove the methylene chloride. This resulted in hardened nanoparticles that were collected by centrifugation at 5000 rpm at 4° C. for 30 min (Beckman-Coulter Avanti-J15R centrifuge). The nanoparticles were re-dispersed in 0.2 wt % of polyvinyl alcohol solution and centrifuged again to remove out residual chemicals. The supernatant was discarded. The cleaned nanoparticles were dispersed in 10 mL of deionized water. About 1 mL of the dispersed nanoparticles was set aside for size and zeta potential characterization. The remaining dispersion was lyophilized. A scanning electron micrograph (SEM) image of the lyophilized nanoparticles taken at 20,000× is shown in FIG. 13. The nanoparticles were determined to have a spherical shape and are singular.

### Example 8: Size and Charge Characterization of Nanoparticles from Example 7

This example demonstrates characterization of size and zeta potential of the nanoparticles formed according to Example 7.

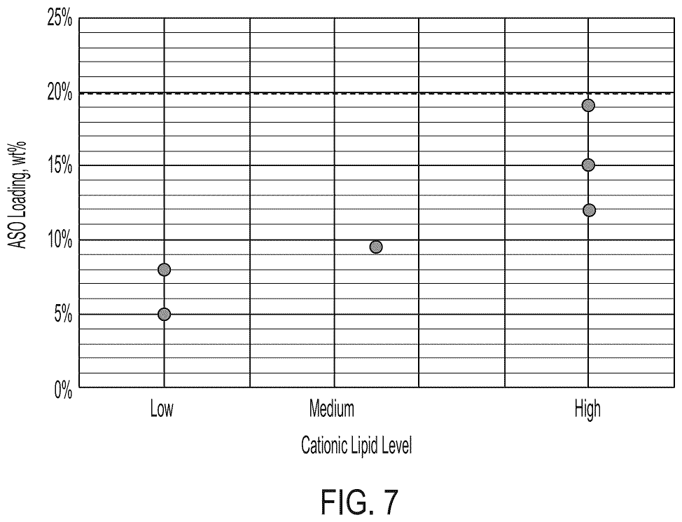

The nanoparticle dispersion described in Example 7 was diluted 5-10 times with di-water before testing. The nanoparticle dispersion was characterized for size and zeta potential was performed utilizing dynamic light scattering with a Zetasizer Ultra (Malvern Panalytical, Malvern United Kingdom). The results are summarized in Table 2. The results are also presented in FIG. 6, which illustrates the relationship between nanoparticle diameter (also referred to as size) and the level of lipid loaded in the nanoparticle. FIG. 8 illustrates the relationship between zeta potential and the level of lipid loaded in the nanoparticle.

### Example 9: Oligonucleotide Loading Characterization of Nanoparticles from Example 7

This example describes characterization assay of oligonucleotide loading of nanoparticles. The results were summarized in Table 2 and in FIG. 7.

Nanoparticles were weighed out and dissolved in 1 mL dimethyl sulfoxide (Sigma Aldrich). The nanoparticle/dimethyl sulfoxide (DMSO) solution was incubated in a water bath at 37° C. for 2 hours to allow the nanoparticles to fully dissolve. To decomplex the oligonucleotide from the lipid salt, 200 μL of the DMSO/nanoparticle solution was added to 500 μL of a 100 mg/mL heparin (Sigma Aldrich) and 100 mM Octyl B-D-glactactoside (Sigma Alrdich) solution in nuclease free, deionized water (Invitrogen). The solution was vortexed for 5 minutes at room temperature, placed on the shaker table for 30 minutes at room temperature, and then vortexed for another 5 minutes before running on an electrophoresis gel for quantification. For the electrophoresis, two oligonucleotide standards were prepared at concentrations of 2 ng/μL and 1 ng/μL, respectively, in nuclease free water (Invitrogen). Oligonucleotide standard solutions include heparin and Octyl B-D-glactactoside in the same concentration that will be in the nanoparticle samples to account for any fluorescence attributed to the reagents. 10 μL of each sample or standard was loaded into a microcentrifuge tube. 2 μL 6× DNA orange loading dye (Invitrogen) was added to each sample and mix well. All 12 μl of each mixture was added to the well of a 10-well 4-20% TBE gel (Invitrogen) in 1× TBE buffer. The gel was run at 250 V for 25 minutes. The gel was removed and stained with 1× Sybr Gold (50,000× stock diluted in 1× TBE buffer, Invitrogen) for 20 minutes with gentle rocking. The gel was imaged using the Image Ready software (Biorad) on the BioRad Gel Doc system. Using the standards of the Image Ready software (Biorad), a standard curve was created and the concentration of the unknown samples were determined from the standard curve. The results are summarized in Table 2 and in FIG. 7 show representative runs, which illustrates the relationship between the loading (wt %) of nucleic acid and the level of lipid loaded in the nanoparticle.

### Example 10: Nanoparticles of Oligo-1 in L-PLA:TMC Copolymer with Medium Level of Lipid Salt DOTAP

This example demonstrates encapsulation of Oligo-1 in L-PLA:TMC copolymer nanoparticles in the presence of medium level of lipid salt DOTAP, specifically 26 wt. % of lipid salt DOTAP and a 3:2 ratio of lipid salt DOTAP to Oligo-1, as described herein.

Nanoparticles were prepared by dissolving approximately 220 mg of L-PLA:TMC 75:25 polymer (as described in Example 1) and 100 mg of lipid salt DOTAP (1,2-dioleoyl-3-trimethylammonium-propane chloride salt, Avanti Polar Lipids Inc) in 8 mL methylene chloride at room temperature. 0.5 mL of 130 mg/ml of Oligo-1 aqueous solution (in DNase/RNase-Free distilled water, Invitrogen) was pipetted into polymer/DOTAP oil solution. Nanoparticles were made and cleaned following the same procedures as described in Example 7. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8, and oligonucleotide loading was measured as described in Example 9. The results are summarized in Table 2. Representative runs are shown in FIGS. 6-8.

### Example 11: Nanoparticles of Oligo-1 in L-PLA:TMC Copolymer with High Level of Lipid Salt DOTAP

This example demonstrates encapsulation of Oligo-1 in L-PLA:TMC copolymer nanoparticles in the presence of high level of lipid salt DOTAP, specifically 32 wt. % of lipid salt DOTAP and a 2:1 ratio of lipid salt DOTAP to Oligo-1, as described herein.

Nanoparticles were prepared by dissolving approximately 160 mg of L-PLA:TMC 75:25 polymer (as described in Example 1) and 100 mg of lipid salt DOTAP (1,2-dioleoyl-3-trimethylammonium-propane chloride salt, Avanti Polar Lipids Inc) in 6 mL methylene chloride at room temperature. 0.5 mL of 100 mg/mL of Oligo-1 aqueous solution (in DNase/RNase-Free distilled water, Invitrogen) was pipetted into polymer/DOTAP oil solution. Nanoparticles were made and cleaned following the same procedures as described in Example 7. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8, and oligonucleotide loading was measured as described in Example 9. The results are summarized in Table 2. Representative runs are shown in FIGS. 6-8.

### Example 12: Nanoparticles of Oligo-1 in L-PLA: TMC Copolymer with Low Level of Lipid Salt DOTMA

This example demonstrates encapsulation of Oligo-1 in L-PLA:TMC copolymer nanoparticles in the presence of low level of lipid salt DOTMA as described herein.

Nanoparticles were prepared by dissolving approximately 160 mg of L-PLA:TMC 75:25 polymer (as described in Example 1) and 50 mg of lipid salt DOTMA (1,2-di-O-octadecenyl-3-trimethylammonium propane chloride salt Avanti Polar Lipids Inc) in 6 mL methylene chloride at room temperature. 0.5 mL of 100 mg/ml of Oligo-1 aqueous solution (in DNase/RNase-Free distilled water, Invitrogen) was pipetted into the polymer/DOTMA oil solution. Nanoparticles were made and cleaned following the same procedures as described in Example 7. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8 and oligonucleotide loading was measured as described in Example 9. The results are summarized in Table 2. Representative runs are shown in FIGS. 6-8.

### Example 13: Nanoparticles of Oligo-1 in L-PLA:TMC Copolymer with High Level of Lipid Salt DOTMA

This example demonstrates encapsulation of Oligo-1 in L-PLA:TMC copolymer nanoparticles in the presence of higher level of lipid salt DOTMA as described herein.

Nanoparticles were prepared by dissolving approximately 160 mg of L-PLA:TMC 75:25 polymer (as described in Example 1) and 100 mg of lipid salt DOTMA (1,2-di-O-octadecenyl-3-trimethylammonium propane chloride salt Avanti Polar Lipids Inc) in 6 mL methylene chloride at room temperature. 1 mL of the 100 mg/ml of Oligo-1 aqueous solution (in DNase/RNase-Free distilled water, Invitrogen) was pipetted into the polymer/DOTAP oil solution. Nanoparticles were made and cleaned following the same procedures as described in Example 7. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8, and oligonucleotide loading was measured as described in Example 9. The results are summarized in Table 2. Representative runs are shown in FIGS. 6-8.

### Example 14: Nanoparticles of Oligo-1 in PLGA Copolymer with Low Level of Lipid Salt DOTAP

This example demonstrates encapsulation of Oligo-1 in PLGA copolymer nanoparticles in the presence of low level of lipid salt DOTAP as comparison.

Nanoparticles were prepared by dissolving approximately 160 mg of PLGA polymer (Resomer® RG 756 S, Poly(D, L-lactide-co-glycolide) purchased from Evonik Industries, Germany) and 50 mg of lipid salt DOTAP (1,2-dioleoyl-3-trimethylammonium propane chloride salt Avanti Polar Lipids Inc) in 6 mL methylene chloride at room temperature. 0.5 mL of the 100 mg/ml of Oligo-1 aqueous solution (in DNase/RNase-Free distilled water, Invitrogen) was pipetted into polymer/DOTAP oil solution. Nanoparticles were made and cleaned following the same procedures as described in Example 7. The cleaned nanoparticles were dispersed in 10 mL of deionized water. About 1 mL of the dispersed particles was set aside for size and zeta potential characterization. The remaining dispersion was lyophilized.

Nanoparticle size and zeta potential were measured as described in Example 8, and oligonucleotide loading was measured as described in Example 9. The results are summarized in Table 2.

### Example 15: Nanoparticles of Oligo-1 in PLGA Copolymer with High Level of Lipid Salt DOTAP

This example demonstrates encapsulation of Oligo-1 in PLGA copolymer nanoparticles in the presence of higher level of lipid salt DOTAP as comparison.

Nanoparticles were prepared by dissolving approximately 160 mg of PLGA polymer (Resomer® RG 756 S, Poly(D,L-lactide-co-glycolide) purchased from Evonik Industries, Germany) and 100 mg of lipid salt DOTAP (1,2-dioleoyl-3-trimethylammonium-propane chloride salt, Avanti Polar Lipids Inc) in 6 mL methylene chloride at room temperature. 1 mL of the 100 mg/mL Oligo-1 aqueous solution (in DNase/RNase-Free distilled water, Invitrogen) was pipetted into the polymer/DOTAP oil solution. Nanoparticles were made and cleaned following the same procedures as described in Example 7. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8, and oligonucleotide loading was measured as described in Example 9. The results are summarized in Table 2. Representative runs are shown in FIGS. 6-8.

TABLE 2

| | | | | | Zeta | Oligo |
|---|---|---|---|---|---|---|
| | | Lipid | Oligo | Particle | potential, | loading, |
| Sample ID | Polymer | Salt | type | size, nm | mv | wt % |
| Example | LT-75 | DOTAP | Oligo-1 | 255 | −41.8 | 8 |
| 7 | | low | | | | |
| Example | LT-75 | DOTAP | Oligo-1 | 261 | 27.7 | 9.5 |
| 10 | | medium | | | | |
| Example | LT-75 | DOTAP | Oligo-1 | 224 | 45.5 | 19 |
| 11 | | high | | | | |
| Example | LT-75 | DOTMA | Oligo-1 | 250 | −45.5 | 7 |
| 12 | | low | | | | |
| Example | LT-75 | DOTMA | Oligo-1 | 240 | 41.1 | 14 |
| 13 | | high | | | | |
| Example | PLGA | DOTAP | Oligo-1 | 209 | 42.2 | 8 |
| 14 | | low | | | | |
| Example | PLGA | DOTAP | Oligo-1 | 226 | 36.4 | 16 |
| 15 | | high | | | | |

### Example 16: Oligonucleotide Release

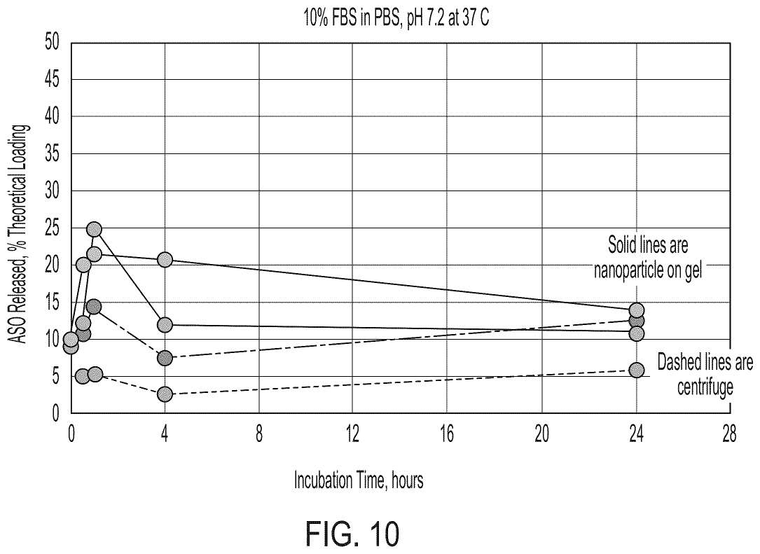

Release of encapsulated oligonucleotide at pH 7 and pH 4, and a summary of the results can be seen in FIGS. 9-10, which show the amount of nucleic acid released as a percentage of theoretical loading and a function of incubation time as discussed herein.

In order to determine if there is a burst release of encapsulated oligonucleotide at pH 7, 1 mL of a 1 mg/ml of oligonucleotide nanoparticle dispersion made according to Example 11 were incubated in a solution of phosphate buffered saline (PBS), supplemented with 10% fetal bovine serum, at 37C for time points of 30 minutes, 1 hour, 4 hours or 24 hours. Samples for analysis were collected in two ways for comparison. In the first method, the incubated samples were taken directly from the test tubes and run on electrophoresis gel (solid lines) as described in Example 9. This method takes into account that nanoparticles are too large to enter the gel, and the quantity found in the gel is only “free” oligonucleotide released in the assay. In the second method, incubated samples were centrifuged at 4° C. for 1 hour at 10,000 rpm (Beckman Coulter) to separate nanoparticles from “free” oligonucleotide (dashed lines). The supernatant of the centrifugation samples were then run on an electrophoresis gel. These two methods were used to determine if either method was artificially separating oligonucleotide from nanoparticles either by force or through the electric current.

To check for encapsulated oligonucleotide release at pH 4, 1 mL of a 1 mg/mL oligonucleotide nanoparticle dispersion made according to from Example 11 were incubated in a phosphate citrate buffer at pH 4. The remaining analysis including incubation and gel electrophoresis is the same as the pH 7 samples.

### Example 17: Nanoparticles of Oligo-1 in L-PLA:TMC and PLA-PEG Copolymer with Lipid Salt DOTAP

This example demonstrates encapsulation of Oligo-1 oligonucleotide in PLA-TMC and PLA-PEG, 2K-5K copolymer nanoparticles in the presence of lipid salt DOTAP.

Nanoparticles were prepared by dissolving approximately 120 mg of L-PLA:TMC 75:25 polymer (as described in Example 1) and 40 mg of PLA-PEG copolymer (Sigma, Methoxy poly(ethylene glycol)-b-poly(D,L-lactide), Mw=2K-5K) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/ml of Oligo-1 oligonucleotide aqueous solution (in DNase/RNase-Free distilled water, Invitrogen) was pipetted into polymer/DOTAP oil solution. The mixture was sonicated for 1 min over ice water bath using a Branson SXF150 sonifier equipped with a 1/4″ tapered probe. Continuous mode was used with C45% of amplitude for sonication. The water-in-oil (W/O) emulsion was placed on a shaker at a speed of 100 rpm/min for overnight. The separated mixture was sonicated again for 1 min over ice water to generate a good dispersion.

Approximately 15 mL of 0.5 wt/v % of cholic acid (Sigma) aqueous solution was then added to the dispersion. The resultant mixture was sonicated for 1 min using the same sonifier and probe over ice water bath. Continuous mode was used with 45% of amplitude for sonication to produce an oil in water emulsion. Additionally, 230 mL of deionized water was added into the emulsion under magnetic stirring at 500 rpm. The emulsion was stirred overnight at room temperature to remove the methylene chloride. This resulted in hardened nanoparticles that were collected by centrifugation at 5000 rpm at 4° C. for 30 min (Beckman Coulter centrifuge). The nanoparticles were re-dispersed in di-water and centrifuged again to remove out residual chemicals. The supernatant was discarded. The cleaned nanoparticles were dispersed in 10 mL of deionized water. About 1 mL of the dispersed nanoparticles was set aside for size and zeta potential characterization. The remaining dispersion was lyophilized.

Nanoparticle size and zeta potential were measured as described in Example 8, and oligonucleotide loading was measured as described in Example 9. The results are summarized in Table 4.

### Example 18: Clean Particles Using a Tangential Flow Filtration System

This example demonstrates a cleaning process used to clean nanoparticles without causing particle coagulation.

Tangential flow filtration technique was used to clean and concentrate nanoparticle emulsions from now on. The system utilized a Pall Corporation (Port Washington, New York) Minimate EVO System, Product ID: OAPMPUNV, with a membrane module MIDIKROS 20 CM 500K MPES 0.5 MM FLL X FLL, Repligen Corporation (Waltham, Massachusetts), Product ID: D02-E500-05-N, mPES membrane, 500 kD, 20 cm. The particle emulsions were first concentrated from 400 mL to 30 mL. 70 mL of di-water was then added into the concentrated emulsion under stirring. The emulsion was concentrated again to 30 mL. This process was repeated twice. The third time the emulsion was concentrated from 100 mL to approximately 15 mL. The cleaned particle emulsion was then pumped into a clean centrifuge tube.

### Example 19: Nanoparticles of Oligo-2 in L-PLA: TMC with Lipid Salt DOTAP

This example demonstrates encapsulation of Oligo-2 oligonucleotide in PLA-TMC copolymer nanoparticles in the presence of lipid salt DOTAP.

Nanoparticles were prepared by dissolving approximately 160 mg of L-PLA:TMC 75:25 polymer (as described in Example 1) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/ml of Oligo-2 oligonucleotide aqueous solution (in DNase/RNase-Free distilled water, Invitrogen) was pipetted into polymer/DOTAP oil solution. The mixture was sonicated for 1 min over ice water bath using a Branson SXF150 sonifier equipped with a 1/4″ tapered probe. Continuous mode was used with C45% of amplitude for sonication. The water-in-oil (W/O) emulsion was placed on a shaker at a speed of 100 rpm/min for overnight. The separated mixture was sonicated again for 1 min over ice water to generate a good dispersion. Approximately 15 mL of 0.5 wt/v % of cholic acid (Sigma) aqueous solution was then added to the dispersion. The resultant mixture was sonicated for 1 min using the same sonifier and probe over ice water bath. Continuous mode was used with 45% of amplitude for sonication to produce an oil in water emulsion. Additionally, 230 mL of deionized water was added into the emulsion under magnetic stirring at 500 rpm. The emulsion was stirred overnight at room temperature to remove the methylene chloride. This resulted in hardened nanoparticles that were cleaned using the method described in Example 18. About 1 mL of the dispersed nanoparticles was set aside for size and zeta potential characterization. The remaining dispersion was lyophilized in the presences of trehalose at the weight ratio of 1:10 particle to trehalose (Sigma) and a 5 mM of histidine buffer with a pH of 4.0 in the particle dispersion.

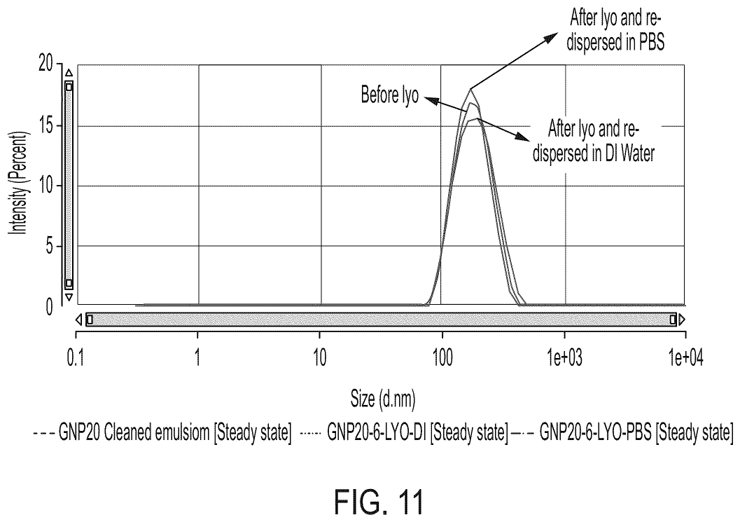

Lyophilized particles were reconstructed in both di-water and PBS. No aggregations were observed in reconstructed dispersion in either di-water and PBS. Particle distributions were showed in FIG. 11.

Nanoparticle size and zeta potential after cleaning and reconstruction were measured as described in Example 8, oligonucleotide loading was measured as described in Example 9. The results are summarized in Table 4.

### Example 20: Nanoparticles of Oligo-3 in L-PLA:TMC with Lipid Salt DOTAP

This example demonstrates encapsulation of Oligo-3 oligonucleotide in PLA-TMC copolymer nanoparticles in the presence of lipid salt DOTAP.

Nanoparticles were prepared by dissolving approximately 160 mg of L-PLA:TMC 75:25 polymer (as described in Example 1) 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/ml of Oligo-3 oligonucleotide aqueous solution (in DNase/RNase-Free distilled water, Invitrogen) was pipetted into polymer/DOTAP oil solution. The mixture was sonicated for 1 min over ice water bath using a Branson SXF150 sonifier equipped with a 1/4″ tapered probe. Continuous mode was used with C45% of amplitude for sonication. The water-in-oil (W/O) emulsion was placed on a shaker at a speed of 100 rpm/min for overnight. The separated mixture was sonicated again for 1 min over ice water to generate a good dispersion. Approximately 15 mL of 0.5 wt/v % of cholic acid (Sigma) aqueous solution was then added to the dispersion. The resultant mixture was sonicated for 1 min using the same sonifier and probe over ice water bath. Continuous mode was used with 45% of amplitude for sonication to produce an oil in water emulsion. Additionally, 230 mL of deionized water was added into the emulsion under magnetic stirring at 500 rpm. The emulsion was stirred overnight at room temperature to remove the methylene chloride. This resulted in hardened nanoparticles that were cleaned using the tangential flow filtration system as describe in example 18. About 1 mL of the dispersed nanoparticles was set aside for size and zeta potential characterization. The remaining dispersion was lyophilized as described in Example 19.

Nanoparticle size and zeta potential were measured as described in Example 8, and oligonucleotide loading was measured as described in Example 9. The results are summarized in Table 4.

### Example 21: Characterization of PEG Surface Density and Coverage on PLA-TMC-PEG Particles

This example describes characterization of PEG content on the surface of nanoparticles made from polymers in Example 2, 3 and 5.

PEGylated nanoparticles were prepared by dissolving approximately 160 mg of L-PLA:TMC-PEG5K 86.4:8.7:4.9 polymer (as described in Example 2) in 6 mL methylene chloride at room temperature. Approximately 15 mL of 0.5 wt/v % of cholic acid (Sigma) aqueous solution was then added to polymer methylene chloride solution. The resultant mixture was sonicated for 1 min using the same sonifier and probe over ice water bath. Continuous mode was used with 45% of amplitude for sonication to produce an oil in water emulsion. Additionally, 230 mL of deionized water was added into the emulsion under magnetic stirring at 500 rpm. The emulsion was stirred overnight at room temperature to remove the methylene chloride. This resulted in hardened nanoparticles that were cleaned using the tangential flow filtration system as described in Example 18.

PEGylated nanoparticles were also prepared from L-PLA:TMC-PEG5K polymer 73.4:21.8:4.8 (as described in Example 3) and L-PLA:TMC-PEG10K polymer 86.5:8.5:5 (as described in Example 5) in the same way as described above. The UV assay that utilized the complexation reactions of PEO with molybdophosphoric acid was used to detect PEG content on the nanoparticles. PEG concentration was found from the difference in absorbance between a blank and the test solution using a calibration curve. According to Koopal et al. (The Effect of Polyethylene Oxide Molecular Weight on Determination of its Concentration in Aqueous Solutions, Talanta, 1982, Vol. 29, p.495-501), this method has no PEO molecular weight dependency. Therefore, the calibration curve was established using PEG of molecular weights of 5K at different concentration (10-500 ug/mL). The blank was obtained from di-water.

The tested PEG content of each batch nanoparticles is listed in Table 3. PEG content on the particles is close to its content in the polymer, which means most of the PEGs are located on the surface of particles.

TABLE 3

| | | PLA | TMC | PEG | | |
|---|---|---|---|---|---|---|
| | | wt % | wt % | wt % | | PEG |
| | Resin in | in | in | in | Particle | wt % on |
| Polymer | Example | resin | resin | resin | size, nm | nanoparticles |
| PLA-TMC- | 2 | 86.4 | 8.7 | 4.9 | 213 | 5.12 |
| PEG5K | | | | | | |
| PLA-TMC- | 3 | 73.4 | 21.8 | 4.8 | 214 | 5.42 |
| PEG5K | | | | | | |
| PLA-TMC- | 5 | 86.5 | 8.5 | 5.0 | 210 | 4.57 |
| PEG10K | | | | | | |

### Example 22: Nanoparticles of Oligo-2 in L-PLA:TMC-PEG Copolymer with Lipid Salt DOTAP

This example demonstrates encapsulation of model Oligo-2 oligonucleotide in PLA-TMC-PEG terpolymer nanoparticles in the presence of lipid salt DOTAP.

Nanoparticles were prepared by dissolving approximately 100 mg of L-PLA:TMC:PEG 73.4:21.8:4.8 terpolymer (as described in Example 3) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/mL of Oligo-2 aqueous solution (in DNase/RNase-Free distilled water, Invitrogen) was pipetted into polymer/DOTAP oil solution. Nanoparticles were made and cleaned following the same procedures as described in Example 18. About 1 mL of the dispersed nanoparticles was set aside for size and zeta potential characterization. The remaining dispersion was lyophilized in the presence of trehalose (Sigma) at a 1:5 particle to trehalose weight ratio. Lyophilized particles were also reconstructed in Di-water and PBS. No aggregation of particles was observed in either di-water or PBS.

Nanoparticle size and zeta potential were measured as described in Example 8, and oligonucleotide loading was measured as described in Example 9 with a little modification. Particles was first dispersed in Acetone and stand at room temperature for at least half hour. DMSO was then added to dissolve particles. DMSO to acetone ratio is 4:1 by volume. A clear particle DMSO/acetone solution was obtained for gel electrophoresis. PEG density on nanoparticle surface was measured as described in Example 21 and may be corrected in accordance with Prophetic Example 28. The results are summarized in Table 4.

### Example 23: Nanoparticles of Oligo-3 in L-PLA:TMC-PEG Copolymer with Lipid Salt DOTAP

This example demonstrates encapsulation of model Oligo-3 oligonucleotide in PLA-TMC-PEG terpolymer nanoparticles in the presence of lipid salt DOTAP.

Nanoparticles were prepared by dissolving approximately 100 mg of L-PLA:TMC:PEG 73.4:21.8:4.8 terpolymer (as described in Example 3) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/ml of Oligo-2 aqueous solution (in DNase/RNase-Free distilled water, Invitrogen) was pipetted into polymer/DOTAP oil solution. Nanoparticles were made and cleaned following the same procedures as described in Example 18. About 1 mL of the dispersed nanoparticles was set aside for size and zeta potential characterization. The remaining dispersion was lyophilized as described in Example 22.

Nanoparticle size and zeta potential were measured as described in Example 8, and oligonucleotide loading was measured as described in Example 9 with a little modification. Particles were first dispersed in Acetone and placed at room temperature for at least half hour. DMSO was then added to dissolve particles. DMSO to acetone ratio is 4:1. A clear particle DMSO/acetone solution was obtained for gel electrophoresis. PEG density on nanoparticle surface was measured as described in Example 21 and may be corrected in accordance with Prophetic Example 28. The results are summarized in Table 4.

### Example 24: Nanoparticles of Oligo-3 in PEG:L-PLA:TMC Copolymer with Lipid Salt DOTMA

This example demonstrates encapsulation of model Oligo-3 oligonucleotide in PLA-TMC-PEG terpolymer nanoparticles in the presence of lipid salt DOTAP.

Nanoparticles were prepared by dissolving approximately 100 mg of L-PLA:TMC:PEG 73.4:21.8:4.8 terpolymer (as described in Example 3) and 100 mg of lipid salt DOTMA (1,2-di-O-octadecenyl-3-trimethylammonium propane chloride salt Avanti Polar Lipids Inc) in 6 mL methylene chloride at room temperature. 1 mL of 100 mg/mL of Oligo-2 aqueous solution (in DNase/RNase-Free distilled water, Invitrogen) was pipetted into polymer/DOTAP oil solution. Nanoparticles were made and cleaned following the same procedures as described in Example 18. About 1 mL of the dispersed nanoparticles was set aside for size and zeta potential characterization. The remaining dispersion was lyophilized described in Example 22.

Nanoparticle size and zeta potential were measured as described in Example 8, and oligonucleotide loading was measured as described in Example 9 with a little modification. Particles was first dispersed in Acetone and were permitted to stand at room temperature for at least half hour. DMSO was then added to dissolve particles. DMSO to acetone ratio is 4:1 by volume. A clear particle DMSO/acetone solution was obtained for gel electrophoresis. PEG density on nanoparticle surface was measured as described in Example 21 may be corrected in accordance with Prophetic Example 28. The results are summarized in Table 4.

### Example 25: Nanoparticles of Oligo-2 in PEG:L-PLA:TMC Copolymer with Lipid Salt DOTAP

This example demonstrates encapsulation of Oligo-2 in PEG-PLA-TMC terpolymer nanoparticles in the presence of lipid salt DOTAP.

This example demonstrates encapsulation of model Oligo-2 oligonucleotide in PLA-TMC-PEG terpolymer nanoparticles in the presence of lipid salt DOTAP.

Nanoparticles were prepared by dissolving approximately 100 mg of L-PLA:TMC:PEG 86.5:8.5:5 terpolymer (as described in Example 5) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/ml of Oligo-2 aqueous solution (in DNase/RNase-Free distilled water, Invitrogen) was pipetted into polymer/DOTAP oil solution. Nanoparticles were made and cleaned following the same procedures as described in Example 18. About 1 mL of the dispersed nanoparticles was set aside for size and zeta potential characterization. The remaining dispersion was lyophilized described in Example 22.

Nanoparticle size and zeta potential were measured as described in Example 8, and oligonucleotide loading was measured as described in Example 9 with a little modification. Particles were first dispersed in Acetone and left to stand at room temperature for at least half hour. DMSO was then added to dissolve particles. DMSO to acetone ratio is 4:1 by volume. A clear particle DMSO/acetone solution was obtained for gel electrophoresis. PEG density on nanoparticle surface was measured as described in Example 21 and may be corrected in accordance with Prophetic Example 28. The results are summarized in Table 4.

### Example 26: Nanoparticles of Oligo-2 in PEG L-PLA:TMC and LT75 Copolymer with Lipid Salt DOTAP

This example demonstrates encapsulation of Oligo-2 in PEG-PLA-TMC and LT75 copolymer nanoparticles in the presence of lipid salt DOTAP.

Nanoparticles were prepared by dissolving approximately 80 mg of L-PLA:TMC:PEG(5K) 82:8:10 polymer (as described in Example 4) and 100 mg of lipid salt DOTAP (1,2-dioleoyl-3-trimethylammonium-propane chloride salt, Avanti Polar Lipids Inc) in 6 mL methylene chloride at room temperature. 1 mL of 100 mg/mL of model Oligo-2 oligonucleotide aqueous solution (in DNase/RNase-Free distilled water, Invitrogen) was pipetted into polymer/DOTAP oil solution. Nanoparticles were made and cleaned following the same procedures as described in Example 16. The cleaned nanoparticles were dispersed in 10 mL of deionized water. About 1 mL of the dispersed nanoparticles was set aside for size and zeta potential characterization. The remaining dispersion was lyophilized described in Example 22.

Nanoparticle size and zeta potential were measured as described in Example 7, and oligonucleotide loading was measured as described in Example 9 with a little modification. Particles was first dispersed in Acetone and were permitted to stand at room temperature for at least half hour. DMSO was then added to dissolve particles. DMSO to acetone ratio is 4:1. A clear particle DMSO/acetone solution was obtained for gel electrophoresis. PEG content on nanoparticle surface was measured as described in Example 21. The high PEG content tested indicating complexation of the reagent with Oligo and/or lipid salt and may be corrected in accordance with Prophetic Example 28. The results were summarized in Table 4.

TABLE 4

| | | | | Particle | Particle | Zeta | Oligo | |
|---|---|---|---|---|---|---|---|---|
| | | Lipid | Oligo | size, | poly- | potential, | loading, | PEG, |
| Sample ID | Polymer | Salt | type | nm | dispersity | mv | wt % | wt % |
| Example 17 | LT-75/PLA- | DOTAP | Oligo-1 | 168 | 0.1 | −21.3 | n/a 2.7* | n/a |
| | PEG 5K-2K | high | | | | | | |
| Example 19 | LT75 | DOTAP | Oligo-2 | 180 | 0.1 | −46.5 | 10 | n/a |
| | | high | | | | | | |
| Example 20 | LT75 | DOTAP | Oligo-3 | 170 | 0.1 | −39.7 | 8.9 | n/a |
| | | high | | | | | | |
| Example 22 | PLA-TMC- | DOTAP | Oligo-2 | 168 | 0.1 | −41 | 9.2 | 14.68% |
| | PEG5K | high | | | | | | |
| Example 23 | PLA-TMC- | DOTAP | Oligo-3 | 182 | 0.15 | −38.2 | 13.9 | 12.02% |
| | PEG5K | high | | | | | | |
| Example 24 | PLA-TMC- | DOTMA | Oligo-3 | 168 | 0.1 | −33 | 7.9 | 22.32% |
| | PEG5K | HIGH | | | | | | |
| Example 25 | PLA-TMC- | DOTAP | Oligo-2 | 174 | 0.1 | −52.2 | 13.8 | 14.68% |
| | PEG10K | high | | | | | | |
| Example 26 | LT75/PLA- | DOTAP | Oligo-2 | 174 | 0.1 | −52.4 | 10.8 | 24.78% |
| | TMC-PEG5K | high | | | | | | |

*Low content due to assay development variability as described in Example 17

These measured PEG wt % values obtained in Examples 22, 23, 24, 25, and 26 may be corrected in accordance with Prophetic Example 28.

### Example 27: Gel Extraction Assay

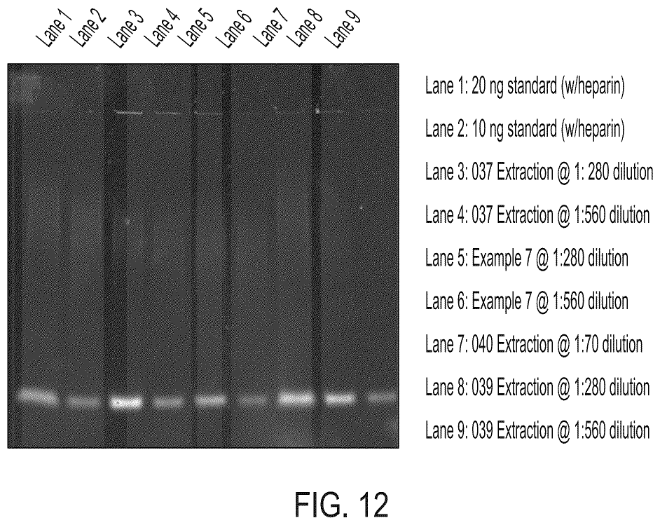

FIG. 12 shows a gel assay of standards, extractions, and specific examples of the nanoparticles as discussed in the Examples above.

### Prophetic Example 28: Determination of Background Reading for PEG Surface Density and Coverage Assay

This example demonstrates a method to determine the background reading of the PEG surface density and coverage assay to allow adjustment of the initial PEG reading with the background reading. The background reading may result from nonspecific binding in the assay, and thus, results in a positive value for PEG when none is present. Performing this correction results in a more accurate, corrected, value for the PEG surface density and coverage of particles produced with PLA-PEG or PLA-TMC-PEG. The background reading is based on particles made of LT-75, ASO, lipid salt and no PEG.

For nanoparticles containing Oligo-2 and DOTAP, nanoparticles of LT-75, Oligo-2, and DOTAP are prepared (as described in Example 19) and are analyzed for PEG surface density and coverage by the assay (as described in Example 21) to determine the background reading for PEG.

To estimate the corrected value for PEG surface density and coverage, the value obtained for the background reading is subtracted from the PEG containing nanoparticle.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A formulation comprising a plurality of bioabsorbable particles, each bioabsorbable particle comprising:
a polymer matrix;
an antisense oligonucleotide (ASO) encapsulated within the polymer matrix; and
a cationic lipid encapsulated within the polymer matrix, where the plurality of bioabsorbable particles contains at least about 1 weight percent of the antisense oligonucleotide (ASO), wherein the polymer matrix comprises trimethylene carbonate (TMC), a copolymer of TMC and polylactic acid (PLA), a terpolymer of TMC, PLA and polyethylene glycol (PEG), a poly(lactic-co-glycolic acid)(PLGA) copolymer of PLA and polyglycolic acid (PGA), or a copolymer of TMC and (PLGA).

2. The formulation of claim 1, wherein the plurality of bioabsorbable particles have an average particle size of less than 300 nm.

3. The formulation of claim 1, wherein the plurality of bioabsorbable particles contain at least about 5 weight percent of the ASO.

4. (canceled)

5. (canceled)

6. (canceled)

7. (canceled)

8. The formulation of claim 1, wherein the ratio of TMC to PLA is about 25:75.

9. (canceled)

10. The formulation of claim 1, further comprising a surface polymer coupled to a surface of each of the bioabsorbable particles.

11. The formulation of claim 10, wherein the surface polymer comprises polyethylene glycol (PEG).

12. (canceled)

13. The formulation of claim 1, where the plurality of bioabsorbable particles contain about 10 weight percent to about 40 weight percent of the cationic lipid.

14. (canceled)

15. The formulation of claim 1, wherein the plurality of bioabsorbable particles comprise nanoparticles.

16. The formulation of claim 1, wherein the plurality of bioabsorbable particles comprise microparticles.

17. (canceled)

18. A method of making a formulation of bioabsorbable particles, the method comprising:
(a) dissolving a structural polymer and a cationic lipid in a solvent;
(b) adding a nucleic acid of 2 to 75 nucleotides in length to the dissolved structural polymer and cationic lipid;
(c) sonicating the mixture of structural polymer, cationic lipid, and nucleic acid in a first sonication to create a water-in-oil emulsion;
(d) adding an aqueous solution to the water-in-oil emulsion;
(e) sonicating the water-in-oil emulsion in a second sonication to produce a water-in-oil-in-water emulsion;
(f) adding water to the water-in-oil-in-water emulsion;
(g) collecting a plurality of particles from the water-in-oil-in-water emulsion; and
(h) cleaning the plurality of particles, wherein the polymer matrix comprises trimethylene carbonate (TMC), a copolymer of TMC and polylactic acid (PLA), a terpolymer of TMC, PLA and polyethylene glycol (PEG), a poly(lactic-co-glycolic acid)(PLGA) copolymer of PLA and polyglycolic acid (PGA), or a copolymer of TMC and (PLGA).

19. The method of claim 18, wherein the cationic lipid and structural polymer are dissolved in a ratio of about 1:4 by mass to about 1:1 by mass.

20. The method of claim 18, wherein the nucleic acid is an antisense oligonucleotide (ASO) and is loaded into the plurality of particles at about 5 weight percent to about 20 weight percent.

21. The method of claim 18, wherein the first sonication and the second sonication are completed in an ice bath.

22. The method according to claim 18, wherein the aqueous solution is a polyvinyl alcohol solution.

23. The method according to claim 18, wherein the step of collecting the plurality of particles from the emulsion is completed through centrifugation.

24. The method according to claim 18, further comprising lyophilizing the oil solution in the presence of a sugar and a buffer.

25. The method according to claim 24, wherein the sugar is trehalose.

26. The method according to claim 24, wherein the buffer is histidine.

27. (canceled)

28. (canceled)

29. (canceled)

30. (canceled)

31. (canceled)

32. (canceled)

33. (canceled)

34. (canceled)
